(19) United States
(12) Patent Application Publication
(10) Pub. No.: US 2019/0128899 A1
(43) Pub. Date: May 2, 2019

(54) METHOD FOR INFERRING ACTIVITY OF A TRANSCRIPTION FACTOR OF A SIGNAL TRANSDUCTION PATHWAY IN A SUBJECT

(71) Applicant: KONINKLIJKE PHILIPS N.V., EINDHOVEN (NL)

(72) Inventors: Anja VAN DE STOLPE, VUGHT (NL); Freek VAN HEMERT, DORDRECHT (NL); Wilhelmus Franciscus Johannes VERHAEGH, Heusden gem. Asten (NL); Reinhold WIMBERGER-FRIEDL, WAALRE (NL); John Cornelius Petrus VAN DER VEN, HELMOND (NL)

(21) Appl. No.: 16/306,091

(22) PCT Filed: Jun. 7, 2017
(86) PCT No.: PCT/EP2017/063744

§ 371 Date: Nov. 30, 2018

(30) Foreign Application Priority Data
Jun. 13, 2016 (EP) 16174158.2

(51) Int. Cl. G01N 33/68; G16B 5/20; G16B 25/10

(52) U.S. Cl. CPC G01N 33/6875; G01N 33/6872; G01N 2800/52; G16B 25/10; G01N 2333/723; G16B 5/20

(57) ABSTRACT

The present invention relates to a method for inferring activity of a transcription factor of a signal transduction pathway in a subject. The method comprises performing (101) a first staining for detecting the transcription factor in cells of a sample of the subject and performing (102) a second staining for detecting a protein encoded by a target gene of the transcription factor in cells of the sample. The method further comprises quantifying (103) cells of the sample that show both a nuclear presence of the transcription factor and a presence of the target gene-encoded protein based on the first staining and the second staining, and inferring (104) the activity of the transcription factor in the subject based on the quantifying. This allows the presented method to more reliably infer the activity of the transcription factor in the subject.

### FIELD OF THE INVENTION

The present invention relates to a method for inferring activity of a transcription factor of a signal transduction pathway in a subject. The present invention further relates to a method for stratifying a subject and to a method for assessing the suitability of a therapy for a subject, the therapy being directed towards a signal transduction pathway, wherein the methods comprise the method for inferring activity the transcription factor of the signal transduction pathway in the subject.

### BACKGROUND OF THE INVENTION

Routine pathology staining in breast cancer today comprises ER (estrogen receptor), PR (progesterone receptor) and HER-2 (human epidermal growth factor receptor 2) staining, for instance, by performing an immunohistochemical (IHC) assay, wherein, in general, one staining is performed per slide. According to clinical diagnostic guidelines, ER and PR are considered to be positive if more than 1 to 10% of the cancer cells in a sample express nuclear ER and PR, respectively, depending on the hospital protocol used. The minimal percentage of cancer cells with nuclear ER and PR staining required for a positive result varies between different countries/centers; in the Netherlands, for instance, the value of 10% is used, whereas in the US already 1% is deemed to signify a positive result. The staining results are used as indications whether the respective signal transduction pathways, for instance, the ER signal transduction pathway and the PR signal transduction pathway, are active in a patient.

At present, ER positive patients are typically treated with hormonal therapy, for instance, neoadjuvant, adjuvant and/or metastatic therapy, wherein the results of the PR staining are not used in guiding the therapy choice. However, with the current practice, about 25 to 50% of the patients are found to not respond to the therapy. A possible reason for this high rate of non-responsiveness may be that in the respective patients the ER signal transduction pathway may actually be inactive despite the ER positive staining result, indicating that the ER staining is insufficiently specific in assessing activity of the ER signal transduction pathway in a patient.

In view of the above, it would generally be desirable to be able to provide improved technologies that allow for a more reliable determination of the activity of a signal transduction pathway in a subject.

### SUMMARY OF THE INVENTION

It is an object of the present invention to provide a method for inferring activity of a transcription factor of a signal transduction pathway in a subject, which allows to more reliably infer the activity of the transcription factor. It is a further object of the present invention to provide a method for stratifying a subject and a method for assessing the suitability of a therapy for a subject, the therapy being directed towards a signal transduction pathway, wherein the methods comprise the method for inferring activity of the transcription factor of the signal transduction pathway in the subject.

In a first aspect of the present invention, a method for inferring activity of a transcription factor of a signal transduction pathway in a subject is presented, wherein the method comprises:
performing a first staining for detecting the transcription factor in cells of a sample of the subject,
performing a second staining for detecting a protein encoded by a target gene of the transcription factor in cells of the sample,
quantifying cells of the sample that show both a nuclear presence of the transcription factor and a presence of the target gene-encoded protein based on the first staining and the second staining, and
inferring the activity of the transcription factor in the subject based on the quantifying.

The present invention is based on the idea that for a cell that shows a nuclear presence of a transcription factor of a signal transduction pathway in the cell, the presence of a protein encoded by a target gene of the transcription factor, either in the nucleus or in the cytoplasma of the cell, can be taken as an additional indicator that the transcription factor is actually active (or “on”), i.e., in the active gene transcribing mode, in the specific cell. Thus, by performing the first staining and the second staining for detecting the transcription factor and the target gene-encoded protein, respectively, in cells of a sample of a subject and by quantifying cells of the sample that show both a nuclear presence of the transcription factor and a presence of the target gene-encoded protein based on the first staining and the second staining, the activity of the transcription factor and, therewith, the activity of the signal transduction pathway, in the subject can be inferred in a more reliable way.

The sample(s) to be used in accordance with the present invention can be an extracted sample, that is, a sample that has been extracted from the subject. Examples of the sample include, but are not limited to, a tissue, cells, blood and/or a body fluid of a subject. It can be, e.g., a sample obtained from a cancer lesion, or from a lesion suspected for cancer, or from a metastatic tumor, or from any other tissue, or from a body cavity in which fluid is present which is contaminated with cancer cells (e.g., pleural or abdominal cavity or bladder cavity), or from other body fluids containing cancer cells, and so forth, preferably via a biopsy procedure or other sample extraction procedure. The cells of which a sample is extracted may also be tumorous cells from hematologic malignancies (such as leukemia or lymphoma). In some cases, the cell sample may also be circulating tumor cells, i.e., tumor cells that have entered the bloodstream and may be extracted using suitable isolation techniques, e.g., apheresis or conventional venous blood withdrawal. Aside from blood, a body fluid of which a sample is extracted may be urine, gastrointestinal contents, or an extravasate. The term “sample”, as used herein, also encompasses the case where e.g. a tissue and/or cells and/or a body fluid of the subject have been taken from the subject and, e.g., have been put on a microscope slide, and where for performing the present invention a portion of this sample is extracted, e.g., by means of Laser Capture Microdissection (LCM), or by scraping off the cells of interest from the slide, or by fluorescence-activated cell sorting techniques. In addition, the term “sample”, as used herein, also encompasses the case where e.g. a tissue and/or cells and/or a body fluid of the subject have been taken from the subject and have been put on a microscope slide, and the claimed method is performed on the slide.

The signal transduction pathway is preferably a nuclear receptor signal transduction pathway. These signal transduction pathways are characterized by an intracellular ligand receptor, which acts as an active transcription factor when a ligand is bound and the receptor translocates to the nucleus of the cell. The active transcription factor subsequently induces transcription of a specific set of targets genes that are translated to proteins. Examples of nuclear receptors are the estrogen receptor, the androgen receptor (AR), the progesterone receptor, the glucocorticoid receptor (GCR), the retinoic acid receptor (RAR), the vitamin D receptor (VDR), and the orphan nuclear receptor.

In an embodiment, the first staining and the second staining are performed on a same slide of the sample. In one approach, the first staining is performed first on the same slide. Thereafter, the stain from the first staining is removed from the same slide, whereupon the second staining is performed on the same slide. In other words: In this approach, the stain from the first staining and the stain from the second staining are not concurrently present on the same slide. This has the advantage that it is not necessary to separate the color information from the first staining and the color information from the second staining in a digital image of the same slide. In another approach, the first staining and the second staining are performed on the same slide such that the stain from the first staining and the stain from the second staining are concurrently present on the same slide. This makes it possible to spare an additional step of removing the stain from the first staining from the same slide.

In another embodiment, the first staining and the second staining are performed on different slides of the sample, wherein the quantifying comprises spatially registering a digital image of the slide of the first staining and a digital image of the slide of the second staining, such that on both slides corresponding cells can be detected. Since the first staining and the second staining are performed on different slides of the sample, they may be performed simultaneously, which may reduce the total time for performing the stainings.

Preferentially, the different slides are obtained from adjacent cross-sections of the sample or from cross-sections of the sample that are located in close proximity to each other in the sample. This helps ensuring that a sufficient number of corresponding cells, i.e., cells that are present are on both slides, are present on the different slides.

It is preferred that the first staining and/or the second staining is/are performed in the form of an assay selected from the group consisting of (i) an immunohistochemistry (IHC) assay, (ii) an immunofluorescent assay, and (iii) another staining assay based on a high affinity binding to the transcription factor or the target gene-encoded protein.

It is further preferred that quantifying comprises:
determining, from a selected population of the cells of the sample, a percentage of cells that show both the nuclear presence of the transcription factor and the presence of the target gene-encoded protein, and/or
determining, from a selected population of the cells of the sample, cells that show the nuclear presence of the transcription factor and determining, from the determined cells, a percentage of cells that also show the presence of the target gene-encoded protein.

In the first variant, the percentage is “directly” determined from the selected population of the cells in the sample. This provides a measurement of the percentage of the cells, from the selected population of the cells of the sample, in which the transcription factor is actually active (or “on”), i.e., in the active gene transcribing mode, wherein the transcription factor could then be considered to be active in the subject, when the percentage exceeds a suitably defined “activity threshold”. The second variant provides a measurement of the percentage of the transcription factor positive cells (i.e., the cells, from the selected population of the cells of the sample that show a nuclear presence of the transcription factor), in which the transcription factor is actually active (or “on”), i.e., in the active gene transcribing mode. Also in this case, the transcription factor could then be considered to be active in the subject, when the percentage exceeds a suitably defined “activity threshold”. Of course, it can also be possible that both percentages are determined and that the inferring of the activity of the transcription factor in the subject is based on both percentages.

It is particularly preferred that the population is a population of cancer cells, in particular, breast cancer cells.

It is preferred that the quantifying comprises analysing at least one digital image of the same slide of the first and the second staining or the digital images of the slide of the first staining and the slide of the second staining using computer-implemented image analysis techniques. By means of such computer-implemented image analysis techniques, the quantifying can be performed in a substantially automated and, in particular, more objective manner that does not rely on the experience of a clinician in interpreting the staining results.

Preferentially, the subject is a medical subject, in particular, a cancer patient, more particularly, a breast cancer patient.

In an embodiment, the signal transduction pathway is the ER-a signal transduction pathway, the transcription factor is ER-a, the target gene is PgR, and the target gene-encoded protein is PR.

In a further aspect of the present invention, a method for assessing the suitability of a therapy for a subject is presented, the therapy being directed towards a signal transduction pathway, wherein the method comprises:
performing the method as defined in any of claims 1 to 10 for inferring activity of a transcription factor of the signal transduction pathway in the subject, and
assessing the suitability of the therapy based on the inferred activity.

In a further aspect of the present invention, a method for stratifying a subject is presented, wherein the method comprises.
performing the method as defined in any of claims 1 to 10 for inferring activity of a transcription factor of a signal transduction pathway in the subject, and
stratifying the subject based on the inferred activity,

In a further aspect of the present invention, a system for use in inferring activity of a transcription factor of a signal transduction pathway in a subject is presented, wherein the system comprises:
a first staining kit for performing a first staining for detecting the transcription factor in cells of a sample of the subject,
a second staining kit for performing a second staining for detecting a protein encoded by a target gene of the transcription factor in cells of the sample,
a quantifying unit for quantifying cells of the sample that show both a nuclear presence of the transcription factor and a presence of the target gene-encoded protein based on the first staining and the second staining, and
optionally, an inferring unit for inferring the activity of the transcription factor in the subject based on the quantifying.

If the system comprises the inferring unit, it can be considered as being a system for inferring activity of a transcription factor of a signal transduction pathway in a subject.

In a further aspect of the present invention, a system for use in assessing the suitability of a therapy for a subject, the therapy being directed towards a signal transduction pathway is presented, wherein the system comprises:
the system for inferring activity of a transcription factor of the signal transduction pathway in the subject as defined in claim 12, and
optionally, an assessing unit for assessing the suitability of the therapy based on the inferred activity.

If the system comprises the assessing unit, it can be considered as being a system for assessing the suitability of a therapy for a subject, the therapy being directed towards a signal transduction pathway.

In a further aspect of the present invention, a system for use in stratifying a subject is presented, wherein the system comprises:
the system for use in inferring activity of a transcription factor of a signal transduction pathway in the subject as defined in claim 12, and
optionally, a stratifying unit for stratifying the subject based on the inferred activity.

If the system comprises the stratifying unit, it can be considered as being a system for stratifying a subject.

In a further aspect of the present invention, a computer program for use in inferring activity of a transcription factor of a signal transduction pathway in a subject is presented, the computer program comprising program code means for carrying out the quantifying step and, optionally, the inferring step of the method for use in inferring activity of the transcription factor in the subject as defined in any of claims 1 to 10, when the computer program is run on a computer.

In a further aspect of the present invention, a computer program for use in assessing the suitability of a therapy for a subject is presented, the therapy being directed towards a signal transduction pathway, the computer program comprising program code means for carrying out the quantifying step and, optionally, the inferring step and/or the assessing step of the method for use in assessing the suitability of the therapy for the subject as defined in claim 11, when the computer program is run on a computer.

In a further aspect of the present invention, a computer program for use in stratifying a subject is presented, the computer program comprising program code means for carrying out the quantifying step and, optionally, the inferring step and/or the stratifying step of the method for use in stratifying the subject as defined in claim 11, when the computer program is run on a computer.

It shall be understood that the method for inferring activity of a transcription factor of a signal transduction pathway in a subject of claim 1, the method for assessing the suitability of a therapy for a subject, the therapy being directed towards a signal transduction pathway, or the method for stratifying a subject of claim 11, and the corresponding systems and computer programs of claims 12 to 14 have similar and/or identical preferred embodiments, in particular, as defined in the dependent claims.

It shall be understood that a preferred embodiment of the present invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows a flowchart exemplarily illustrating a first embodiment of a method for inferring activity of a transcription factor of a signal transduction pathway in a subject,

FIG. 2 shows a flowchart exemplarily illustrating a second embodiment of a method for inferring activity of a transcription factor of a signal transduction pathway in a subject,

FIG. 3 shows a flowchart exemplarily illustrating a third embodiment of a method for inferring activity of a transcription factor of a signal transduction pathway in a subject,

FIG. 4 shows a flowchart exemplarily illustrating an embodiment of a method for assessing the suitability of a therapy for a subject, the therapy being directed towards a signal transduction pathway,

FIG. 5 shows a flowchart exemplarily illustrating an embodiment of a method for stratifying a subject,

FIG. 6 shows schematically and exemplarily an embodiment of a system for use in inferring activity of a transcription factor of a signal transduction pathway in a subject,

FIG. 7 shows schematically and exemplarily an embodiment of a system for use in assessing the suitability of a therapy for a subject, the therapy being directed towards a signal transduction pathway,

FIG. 8 shows schematically and exemplarily an embodiment of a system for use in stratifying a subject,

FIG. 9 shows schematically and exemplarily a detection of the outlines of cells and their nuclei in a digital image of a slide of a sample,

FIG. 10 shows a comparison of the progression of the ER-a signal and the PR signal measured in an MCF7 breast cancer cell line sample upon estrogen deprivation for 48 hours and subsequent estradiol stimulation for up to 16 hours, and

FIG. 11 shows results of ER and PR quantification experiments performed with 39 breast cancer samples.

### DETAILED DESCRIPTION OF EMBODIMENTS

In the drawings, like or corresponding reference numerals refer to like or corresponding parts and/or elements.

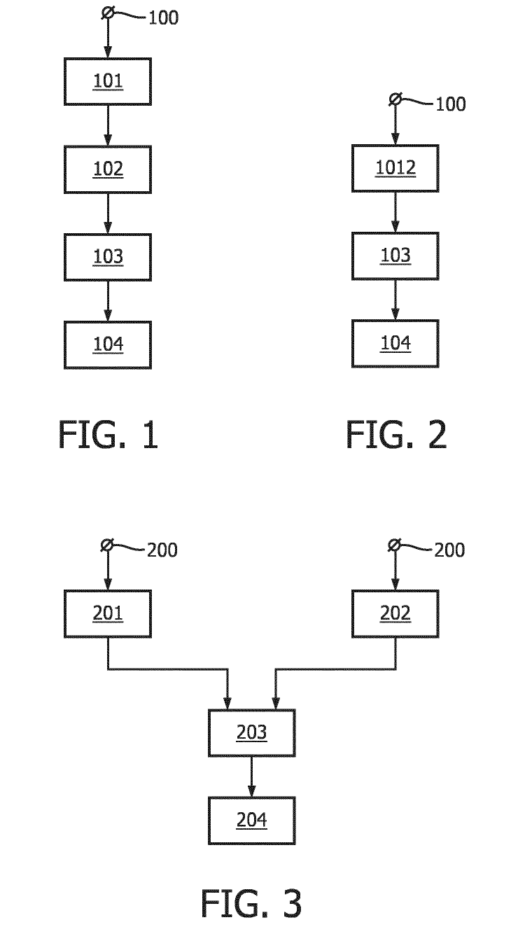

In the following, a first embodiment of a method for inferring activity of a transcription factor of a signal transduction pathway, here, the ER-a signal transduction pathway, in a subject will exemplarily be described with reference to a flowchart shown in FIG. 1. The subject, here, is a medical subject, in particular, a cancer patient, more particularly, a breast cancer patient.

In step 101, a first staining for detecting the transcription factor, here, ER-α, in cells of a sample of, in this example, the tissue of the breast cancer patient is performed. In step 102, a second staining for detecting a protein, here, PR, encoded by a target gene, here, PgR, of the transcription factor, i.e., ER-α, in cells of the sample is performed. The first staining and the second staining are performed on a same slide of the sample. Here, the first staining and the second staining are both performed in the form of an immunohistochemistry (IHC) assay.

In this embodiment, the first staining is performed first on the same slide (step 101). Thereafter, the stain from the first staining is removed from the same slide, whereupon the second staining is performed on the same slide (step 102). In other words: in this embodiment, the stain from the first staining and the stain from the second staining are not concurrently present on the same slide.

In step 103, cells of the sample that show both a nuclear presence of ER-a and a (nuclear and/or cytoplasmic) presence of the PgR-encoded protein PR are quantified based on the first staining and the second staining. In this embodiment, this is done by analyzing digital images of the same slide with the stain from the first staining and with the stain from the second staining using computer-implemented image analysis techniques. The digital images can be obtained, for instance, by scanning the stained same slide using a suitable scanning apparatus or device.

In more detail, the outlines of cells and their nuclei are detected in the digital images of the same slide based on the color information from the first staining and/or the color information from the first staining—possibly, in combination with a further H&E (Haematoxylin and Eosin) staining—using computer-implemented detection techniques as described, for instance, in J. P. Vink et al., “Efficient nucleus detector in histopathology images”, in Journal of Microscopy, Vol. 249, No. 2, 2013, pages 124 to 135.

Based on the detected outlines of cells and their nuclei, the quantifying then comprises, in this embodiment, determining an intensity of the color information from the first staining in the nuclei of cells and determining an intensity of the color information from the second staining in the nuclei and the cytoplasms of cells. This process can include different processing steps, such as the use of filtering operations, the use of morphological operations, or the like. Cells that show both a nuclear presence of ER-α (transcription factor) and a (nuclear and/or cytoplasmic) presence of the PgR-encoded protein PR are then suitably detected by thresholding the intensities. This is done, for instance, by detecting cells in which both the intensity of the color information from the first staining in the nucleus of the cell and the intensity of the color information from the second staining in the nucleus or the cytoplasm of the same cell exceed a predefined threshold. Alternatively, instead of predefining such threshold, it can be set by a background staining in non-cancerous parts of the same slide, in which the cells do not show a nuclear presence of ER-α and/or and a (nuclear and/or cytoplasmic) presence of the PgR-encoded protein PR, or the first staining and/or the second staining can be normalized against such a background staining. It is noted that depending on the specifics of the process, such as the wavelengths of the stains from the first staining and the second staining, the scanning apparatus or device used to obtain the digital images, et cetera, the predefined threshold may be the same for both the color information from the first staining and the color information from the second staining, or different thresholds may be predefined or set. The quantifying, here, further comprises determining a percentage of cells that show both a nuclear presence of ER-α and a (nuclear and/or cytoplasmic) presence of the PgR-encoded protein PR. In a first variant, the percentage is “directly” determined from a selected population of the cells in the sample, wherein the population is, for instance, a population of breast cancer cells. In a second variant, the percentage can be determined by determining, from a selected population of the cells of the sample (e.g., a population of breast cancer cells), cells that show the nuclear presence of ER-α and determining, from the determined cells, a percentage of cells that also show the (nuclear and/or cytoplasmic) presence of the PgR-encoded protein PR.

Since the digital images, i.e., the digital image of the same slide with the stain from the first staining and the digital image of same slide with the stain from the second staining, are separately obtained from the same slide, it may be necessary that the quantifying comprises spatially registering the digital images with each other, such that the color information from the first staining and the second staining can be overlaid to enable identification of cells in which both stainings co-locate in the same cell. Such spatial registration can be achieved using computer-implemented image processing techniques, such as those described in D. Mueller et al., “Real-time deformable registration of multi-modal whole slides for digital pathology”, in Computerized Medical Imaging and Graphics, No. 35, 2011, pages 542 to 556. Since the digital images are obtained from the same slide, the spatial registration can be almost perfect.

In step 104, the activity of the transcription factor, here, ER-α, in the breast cancer patient is inferred based on the quantifying, here, using a computer-implemented algorithm. Based on the conventional clinically used ER staining, where ER is considered to be positive if more than 1 or 10% of the cancer cells in a sample are positively stained (over a threshold, depending on the hospital protocol used), the same value of 1 or 10% may be used as an “activity threshold”, but instead of considering all cells, from the selected population of the cells of the sample (e.g., a population of breast cancer cells) that show a nuclear presence of ER-α only those cells are considered that also show a (nuclear and/or cytoplasmic) presence of the PgR-encoded protein PR (cf. above). This provides a measurement of the percentage of the cells, from the selected population of the cells of the sample (e.g., a population of breast cancer cells), in which the transcription factor, here, ER-α, is actually active (or “on”), i.e., in the active gene transcribing mode, wherein the transcription factor (ER-α) would then be considered to be active in the breast cancer patient, when the percentage exceeds the activity threshold of 1 or 10%. Alternatively, if the percentage is determined in the quantifying by determining, from a selected population of the cells of the sample (e.g., a population of breast cancer cells), cells that show the nuclear presence of ER-α and determining, from the determined cells, a percentage of cells that also show the (nuclear and/or cytoplasmic) presence of the PgR-encoded protein PR (cf. above), this provides a measurement of the percentage of the ER-α positive cells (i.e., the cells, from the selected population of the cells of the sample (e.g., a population of breast cancer cells) that show a nuclear presence of ER-α), in which the transcription factor, here, ER-α, is actually active (or “on”), i.e., in the active gene transcribing mode. Also in this case, the transcription factor (ER-α) could then be considered to be active in the breast cancer patient, when the percentage exceeds an activity threshold of 1 or 10%. Of course, other values of the activity threshold may be used for deciding whether or not the transcription factor, here, ER-α, could be considered to be active in the breast cancer patient. Moreover, it can also be possible that both percentages are determined and that the inferring of the activity of the transcription factor, here, ER-α, in the breast cancer patient is based on both percentages.

In the following, a second embodiment of a method for inferring activity of a transcription factor of a signal transduction pathway, here, the ER-α signal transduction pathway, in a subject will exemplarily be described with reference to a flowchart shown in FIG. 2. The subject, here, is again a medical subject, in particular, a cancer patient, more particularly, a breast cancer patient.

The second embodiment is substantially similar to the first embodiment described with reference to the flowchart shown in FIG. 1. In particular, also in this embodiment, a first staining for detecting the transcription factor, here, ER-α, in cells of a sample of, in this example, the tissue of the breast cancer patient is performed, and a second staining for detecting a protein, here, PR, encoded by a target gene, here, PgR, of the transcription factor, i.e., ER-α, in cells of the sample is performed. However, while also in this case the first staining and the second staining are performed on a same slide of the sample, they are performed, in step 1012, such that the stain from the first staining and the stain from the second staining are concurrently present on the same slide. Here, the first staining and the second staining are both performed in the form of an immunohistochemistry (IHC) assay.

Also in this embodiment, cells of the sample that show both a nuclear presence of ER-α and a (nuclear and/or cytoplasmic) presence of the PgR-encoded protein PR are quantified based on the first staining and the second staining (step 103). Here, this is done by analyzing a digital image of the same slide with the stain from the first staining and with the stain from the second staining using computer-implemented image analysis techniques. The digital image can be obtained, for instance, by scanning the stained same slide using a suitable scanning apparatus or device.

Since the stain from the first staining and the stain from the second staining are concurrently present on the same slide, their wavelengths are suitably selected such that they are substantially non-overlapping and the color information from the first staining and the color information from the second staining is separated in the digital image using a computer-implemented color deconvolution technique as described, for instance, in A. C. Ruifrok and D. A. Johnston, “Quantification of histochemical staining by color deconvolution”, in Analytical and Quantitative Cytology and Histology, Vol. 23, No. 4, 2001, pages 291 to 299, in M. Macenko et al., “A method for normalizing histology slides for quantitative analysis”, in Proceedings of IEEE International Symposium on Biomedical Imaging: From Nano to Macro, Boston, Mass., USA, 2009, pages 1107 to 1110, in M. Niethammer et al., “Appearance normalization in histology slides”, in Proceedings of First International Workshop on Machine Learning in Medical Imaging, Beijing, China, 2010, pages 58 to 66, or in J. P. Vink et al. (ibid).

Based on the separated color information from the first staining and/or the separated color information from the second staining—possibly, in combination with a further H&E (Haematoxylin and Eosin) staining—, the quantifying (step 103) and the inferring of the activity of the transcription factor, here, ER-α, in the breast cancer patient based on the quantifying (step 104) are then performed substantially as described above for the first embodiment.

In the following, a third embodiment of a method for inferring activity of a transcription factor of a signal transduction pathway, here, the ER-α signal transduction pathway, in a subject will exemplarily be described with reference to a flowchart shown in FIG. 3. The subject, here, is again a medical subject, in particular, a cancer patient, more particularly, a breast cancer patient.

The third embodiment is substantially similar to the first and second embodiment described with reference to the flowcharts shown in FIGS. 1 and 2. In particular, also in this embodiment, a first staining for detecting the transcription factor, here, ER-α, in cells of a sample of, in this example, the tissue of the breast cancer patient is performed, and a second staining for detecting a protein, here, PR, encoded by a target gene, here, PgR, of the transcription factor, i.e., ER-α, in cells of the sample is performed. However, in this case the first staining and the second staining are performed, in steps 201 and 202, on different slides of the sample. Here, the different slides are obtained from adjacent cross-sections of the sample and the first staining and the second staining are both performed in the form of an immunohistochemistry (IHC) assay. Instead of obtaining the different slides from adjacent cross-sections of the sample, it can also be possible that the different slides are obtained from cross-sections of the sample that are located in close proximity to each other in the sample. In this case, the distance between the cross-sections should not be larger than, for instance, five cross-sections of 4 microns each.

Also in this embodiment, cells of the sample that show both a nuclear presence of ER-α and a (nuclear and/or cytoplasmic) presence of the PgR-encoded protein PR are quantified based on the first staining and the second staining (step 203). Here, this is done by analyzing digital images of the slide with the stain from the first staining and the slide with the stain from the second staining using computer-implemented image analysis techniques. The digital images can be obtained, for instance, by scanning the stained slides using a suitable scanning apparatus or device.

Since the digital images, i.e., the digital image of the slide with the stain from the first staining and the digital image of the slide with the stain from the second staining, are obtained from different slides obtained from adjacent cross-sections of the sample, it is in general necessary that the quantifying comprises spatially registering the digital images with each other. Such spatial registration can be achieved using computer-implemented image processing techniques, such as those described in D. Mueller et al. (ibid). Whatever method used, it should enable alignment of the digital images of the two slides, such that on both slides corresponding cells can be detected, making it possible to determine and quantify cells in which the first staining and the second staining are present in the same cell. Since the digital images are obtained from different slides obtained from adjacent cross-sections of the sample, the spatial registration will not be perfect, because the slide of the first staining and the slide of the second staining will comprise—at least partly—different cells. To account for this issue, cells for which it is not clear that they are present in both slides may not be used in the quantifying (step 203) and the inferring of the activity of the transcription factor, here, ER-α, in the breast cancer patient based on the quantifying (step 204), which are otherwise performed substantially as described above for the first and second embodiment.

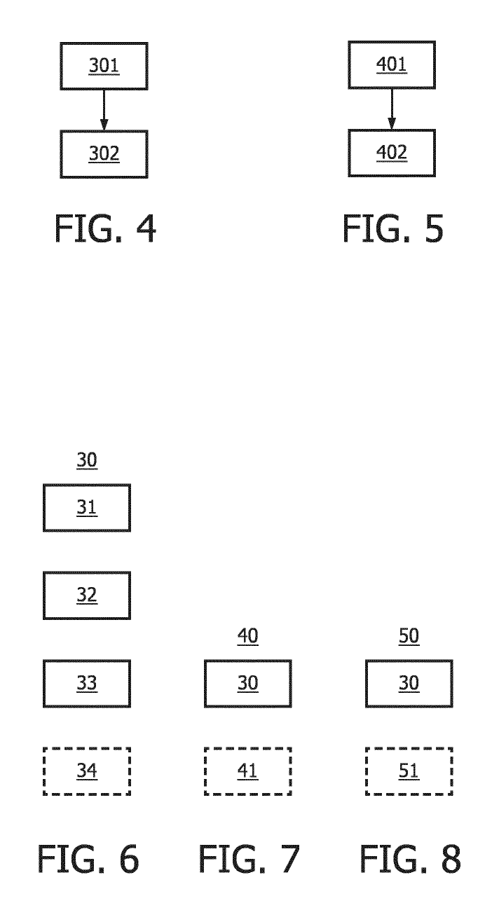

In the following, an embodiment of a method for assessing the suitability of a therapy for a subject, the therapy being directed towards a signal transduction pathway, here, the ER-α signal transduction pathway, will exemplarily be described with reference to a flowchart shown in FIG. 4. The subject, here, is again a medical subject, in particular, a cancer patient, more particularly, a breast cancer patient.

In step 301, the method as described with reference to any of the flowcharts shown in FIGS. 1 to 3 is performed for inferring activity of the transcription factor, here, ER-α, in the breast cancer patient.

In step 302, the suitability of the therapy is determined based on the inferred activity. The therapy, in this embodiment, is hormonal therapy (also called “endocrine therapy”), which can reduce in different ways estradiol signaling through the ER-α signal transduction pathway and which may consist of drugs directed “directly” towards the transcription factor, i.e., ER-α, like Tamoxifen, or drugs which interfere with estradiol ligand production, like aromatase inhibitors, or drugs which promote ER-α degradation. In more detail: Aromatase inhibitor drugs (e.g., Exemestane) interfere with estradiol production, resulting in reduced levels of the ligand that activates the ER-α signal transduction pathway. In contrast, estrogen receptor (ER)-inhibitory drugs (e.g., Tamoxifen) in general compete with estradiol for its binding place on the estrogen receptor, and thus interfere with activation. Finally, a rather new drug, Fulvestrant, increases ER-α degradation.

By basing the determination of the suitability of the therapy, here, hormonal therapy, on the inferred activity of the transcription factor, here, ER-α, in the breast cancer patent, if the transcription factor (ER-α) is inferred to be active, the likelihood of response to the therapy is expected to be higher than is currently the case with the conventional interpretation of ER (and PR) staining in the clinic, which means that the therapy response prediction gains in specificity, while retaining sensitivity. This is clinically highly relevant, since in case the breast cancer patient is predicted to be a non-responder based on the inferred activity of the transcription factor, here, ER-α, another more suitable and effective therapy can be chosen in time, thus preventing progression of cancer in the presence of an ineffective therapy and preventing unnecessary side effects (as well as unnecessary costs). For instance, based on the above-discussed activity threshold of 1 or 10%, it is expected that if the percentage of the cells, from the selected population of the cells of the sample (e.g., a population of breast cancer cells), in which the transcription factor, here, ER-α, is actually active (or “on”), i.e., in the active gene transcribing mode, exceeds the activity threshold, the breast cancer patient's likelihood of response to hormonal therapy is greater than 60% (for the 1% activity threshold) and greater than 80% (for the 10% activity threshold), respectively. The same—or even higher—likelihoods are expected if the same activity threshold of 1 or 10% is used and the percentage is determined in the quantifying by determining, from a selected population of the cells of the sample (e.g., a population of breast cancer cells), cells that show the nuclear presence of ER-α and determining, from the determined cells, a percentage of cells that also show the (nuclear and/or cytoplasmic) presence of the PgR-encoded protein PR (cf. above). In this respect, it shall be noted that clinical calibration of the percentage of cells with an active transcription factor, here, ER-α, required for an adequate hormonal therapy response will preferably be based on evaluations in appropriate breast cancer patient cohorts.

So, in essence, if the transcription factor, here, ER-α, is inferred to be active in the breast cancer patent, drugs like Tamoxifen or aromatase inhibitors are likely to be effective in suppressing tumor growth, except in the case of an activating mutation in ER-α, in which case Fulvestrant is the therapy of choice. A definitive therapy choice can then be made by an additional assessment of the activating ER-α mutation by, for instance, DNA or RNA sequencing.

On the other hand, if the transcription factor, here, ER-α, is inferred to be inactive (or “off”), i.e., not in the active gene transcribing mode, in the breast cancer patent, it is likely that the breast cancer patient will not respond to hormonal therapy (e.g., Tamoxifen or aromatase inhibitors). In this case, alternative therapies can, for instance, consist of chemotherapy or alternative targeted therapies or immunotherapy or combination therapy.

In the following, an embodiment of a method for stratifying a subject will exemplarily be described with reference to a flowchart shown in FIG. 5. The subject, here, is again a medical subject, in particular, a cancer patient, more particularly, a breast cancer patient.

In step 401, the method as described with reference to any of the flowcharts shown in FIGS. 1 to 3 is performed for inferring activity of a transcription factor of a signal transduction pathway, here, the ER-α signal transduction pathway, in the breast cancer patient.

In step 402, the breast cancer patient is stratified based on the inferred activity. For instance, if the transcription factor, here, ER-α, is inferred to be active (or “on”), i.e., in the active gene transcribing mode, in the breast cancer patent, the breast cancer patient can be stratified for hormonal treatment, and if the transcription factor, here, ER-α, is inferred to be inactive (or “off”), i.e., not in the active gene transcribing mode, the breast cancer patient can be stratified for an alternative therapy, for instance, chemotherapy or an alternative targeted therapy or immunotherapy or combination therapy. The respective stratification decisions can be the same as the decisions made in the method for assessing the suitability of a therapy for a subject described with reference to the flowchart shown in FIG. 4.

In the following, an embodiment of a system for use in inferring activity of a transcription factor of a signal transduction pathway, here, the ER-α signal transduction pathway, in a subject is schematically and exemplarily shown in FIG. 6. The subject, here, is again a medical subject, in particular, a cancer patient, more particularly, a breast cancer patient.

The system 30 comprises a first staining kit 31 for performing a first staining for detecting the transcription factor, here, ER-α, in cells of a sample of, in this example, the tissue of the breast cancer patient and a second staining kit 32 for performing a second staining for detecting a protein, here, PR, encoded by a target gene, here, PgR, of the transcription factor, i.e., ER-α, in cells of the sample. The system 30 further comprises a quantifying unit 33 for quantifying cells of the sample that show both a nuclear presence of ER-α and a (nuclear and/or cytoplasmic) presence of the PgR-encoded protein PR based on the first staining and the second staining and, optionally, an inferring unit 34 for inferring the activity, here, ER-α, of the transcription factor in the breast cancer patient based on the quantifying.

The system 30 is preferably adapted to perform the method as described with reference to any of the flowcharts shown in FIGS. 1 to 3.

In the following, an embodiment of a system for use in assessing the suitability of a therapy for a subject, the therapy being directed towards a signal transduction pathway, here, the ER-α signal transduction pathway, is schematically and exemplarily shown in FIG. 7. The subject, here, is again a medical subject, in particular, a cancer patient, more particularly, a breast cancer patient.

The system 40 comprises a system 30 as described with reference to FIG. 6 and, optionally, an assessing unit 41 for assessing the suitability of the therapy based on the inferred activity.

The system 40 is preferably adapted to perform the method as described with reference to the flowchart shown in FIG. 4.

In the following, an embodiment of a system for use in stratifying a subject, is schematically and exemplarily shown in FIG. 8. The subject, here, is again a medical subject, in particular, a cancer patient, more particularly, a breast cancer patient.

The system 50 comprises a system 30 as described with reference to FIG. 6 and, optionally, a stratifying unit 51 for stratifying the subject based on the inferred activity.

The system 50 is preferably adapted to perform the method as described with reference to the flowchart shown in FIG. 5.

In the first to third embodiment of a method for inferring activity of a transcription factor of a signal transduction pathway, here, the ER-α signal transduction pathway, in a subject described with reference to FIGS. 1 to 3, the method may be preceeded by an appropriate sample processing (step 100; step 200). For instance, the sample can be obtained from fresh or paraffin-embedded tissue/cell material that is processed to be fixed and deposited on a microscope slide, to be stained according to standard IHC or immunofluorescence staining protocols.

While in the first to third embodiment of a method for inferring activity of a transcription factor of a signal transduction pathway, here, the ER-α signal transduction pathway, in a subject described with reference to FIGS. 1 to 3, the first staining and the second staining are both performed in the form of an immunohistochemistry (IHC) assay, in other embodiments only one of the first staining and the second staining can be performed in the form of an IHC assay while the other one of the first staining and the second staining can be performed as an immuofluorescent assay. Alternatively, both the first staining and the second staining can be performed as an immunofluorescent assay or the first staining and/or the second staining can be performed as another staining assay based on a high affinity binding to the transcription factor, here, ER-α, or the target gene-encoded protein PR (target gene, PgR), for instance, a staining assay based on antibodies with colloids (gold beads), a staining assay with isotopes for photographic development or a staining assay based on enhanced chemiluminescence (ECL).

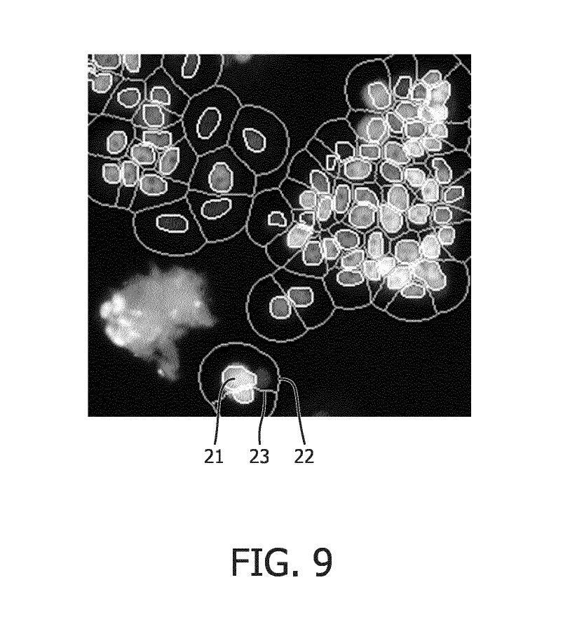

In the first to third embodiment of a method for inferring activity of a transcription factor of a signal transduction pathway, here, the ER-α signal transduction pathway, in a subject described with reference to FIGS. 1 to 3, an additional DAPI (diamidino phenylindole) staining can be performed to show the nuclei of the cells in order to ease computer-implemented image analysis performed as part of the quantifying. In this respect, FIG. 9 shows schematically and exemplarily a detection of the outlines of cells and their nuclei in a digital image of a slide of a sample. In this example, a computer-implemented algorithm is used to detect cells based on the nuclei present in the DAPI (blue) channel of the digital image. One example of a nucleus with an identified outline based on the DAPI staining is designated in FIG. 9 by the reference numeral 21. In the absence of a membrane staining, the nucleus 21 serves as a kernel from which a virtual membrane 22 is grown outwards until a given surface area is reached or the membrane 22 hits other membranes, here, for instance, the membrane 23. The membrane 22, together with the defined nucleus 21, provides a nuclear and a cytoplasmic compartment that are used for quantifying stainings in other fluorescence channels. For example, in the staining shown in FIG. 9, an average nuclear staining in a green fluorescence channel can be quantified per cell in order to quantify cells of the sample that show a nuclear presence of the transcription factor, here, ER-α.

While in the first to third embodiment of a method for inferring activity of a transcription factor or a signal transduction pathway, here, the ER-α signal transduction pathway, in a subject described with reference to FIGS. 1 to 3, the first staining is described as being performed first, i.e., before the second staining, in other embodiments the second staining can be performed first, i.e., before the first staining. For instance, in the first embodiment described with reference to FIG. 1, the second staining can be performed first on the same slide and the first staining can be performed only after the stain from the second staining has been removed from the same slide. In other words: The terms “first” and “second” in the expressions “first staining” and “second staining” are only used for distinguishing the two stainings from each other; they do not imply a relative order in with the two stainings are performed.

As mentioned above, in the conventional clinically used ER staining, nuclear ER staining in a cell is considered to indicate an active ER signal transduction pathway in that cell. This assumption, however, is not necessarily true as is shown in the following for a sample from the MCF-7 breast cancer cell line. Here, three slices were cut from a tissue block of the breast cancer patient's sample, wherein the first and the last slice served as controls. For the middle slice, a first staining was performed for detecting a transcription factor, here, ER-α, in cells of the sample, and a second staining was performed for detecting a protein, here, PR, encoded by a target gene, here, PgR, of the transcription factor, i.e., ER-α, in cells of the sample. In more detail: Both the first staining and the second staining were performed in the form of an immunofluorescent assay on a same slide of the sample, such that the stain from the first staining and the stain from the second staining were concurrently present on the same slide. Since the stain from the first staining and the stain from the second staining were concurrently present on the same slide, their wavelengths were suitably selected such that they are substantially non-overlapping.

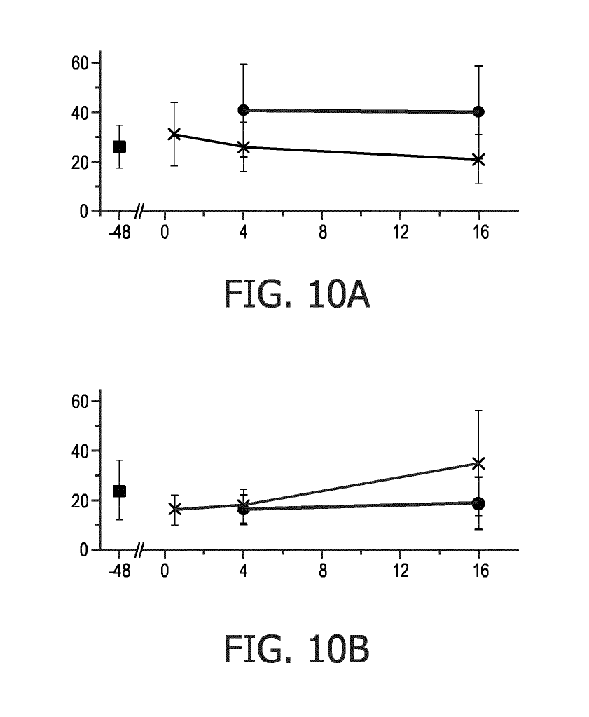

FIG. 10 shows a comparison of the progression of the ER-α signal (top graph) and the PR signal (bottom graph) measured in an MCF7 breast cancer cell line sample upon estrogen deprivation for 48 hours and subsequent estradiol stimulation for up to 16 hours. In the graphs, the horizontal axis indicates the time in hours and the vertical axis indicates the intensity of the respective protein expression signals in the nuclei of the MCF7 cells measured using specific antibodies directed to these proteins. As can be seen from the top graph, after the 48 hour period of estrogen deprivation, the MCF7 cells show an increased ER-α signal (marked with the crosses) with respect to normal culture conditions (marked with the black square). Upon stimulation with estradiol, the ER signal decreases with respect to the control (no stimulation, marked with the black dots). The bottom graph shows that the PR signal (marked with the crosses), on the other hand, increases its nuclear signal in response to the ER-α signal transduction pathway activation with estradiol compared to the control (unstimulated, marked with the black dots). The PR signal increases to levels above that of normal culture conditions (marked with the black square) after 16 hours in the presence of estradiol. This shows that, indeed, the specific stimulation of the ER pathway results in an increased production of the PR protein. The fact that ER itself is down-regulated is expected and has been described before in literature (Borrás M. et al., “Estradiol-induced down-regulation of estrogen receptor. Effect of various modulators of protein synthesis and expression”, J Steroid Biochem Mol Biol, Vol. 48, No. 4, 1994, pages 325 to 336).

In order to verify the ability of the methods according to the invention to allow for a reliable inference of a transcription factor, here, ER-α, and, therewith, the activity of the signal transduction pathway, here, the ER-α signal transduction pathway, in a subject, the activity of the signal transduction pathway, here, the ER-α signal transduction pathway, was also inferred by means of the method described in Verhaegh W. et al., “Selection of personalized patient therapy through the use of knowledge-based computational models that identify tumor-driving signal transduction pathways”, Cancer Research, Vol. 74, No. 11, 2014, pages 2936 to 2945, wherein the method was slightly adapted to use qPCR data instead of expression microarray data for the signal transduction pathway activity interpretation. The following Table 1 then shows the inferred probability (third column) and the log 2 of the probability (fourth column) that the ER-α signal transduction pathway is indeed active (or “on”), i.e., in the active gene transcribing mode. As can be seen from the table, the log 2 of the probability that the ER-α signal transduction pathway is active in a breast cancer sample that shows both a nuclear presence of the transcription factor, here, ER-α and a (nuclear and/or cytoplasmic) presence of the PgR-encoded protein PR (abbreviated here as “ER+PR+”), is inferred to be 3.2, indicating activity of the ER-α signal transduction pathway in this sample. In contrast, for a breast cancer sample in which only the ER-α staining is positive (abbreviated here as “ER+PR−”), the log 2 of the probability is inferred to be −0.676, indicating inactivity of the ER-α signal transduction pathway in this part. This result confirms that a more reliable inference of the activity of a transcription factor, here, ER-α, and, therewith, the activity of the signal transduction pathway, here, the ER-α signal transduction pathway, in a subject may be achieved when considering cells of a sample of a subject that show both a nuclear presence of the transcription factor, here, ER-α and a (nuclear and/or cytoplasmic) presence of a protein, here, PR, encoded by a target gene, here, PgR, of the transcription factor, i.e., ER-α.

TABLE 1
Likelihood that the ER-α signal transduction pathway is active.

| Sample | Probalility ER-α “on” | Log2 of probability ER-α “on” |
|---|---|---|
| ER+PR− | 0.38498161 | −0.675840018 |
| ER+PR+ | 0.901885007 | 3.200397975 |

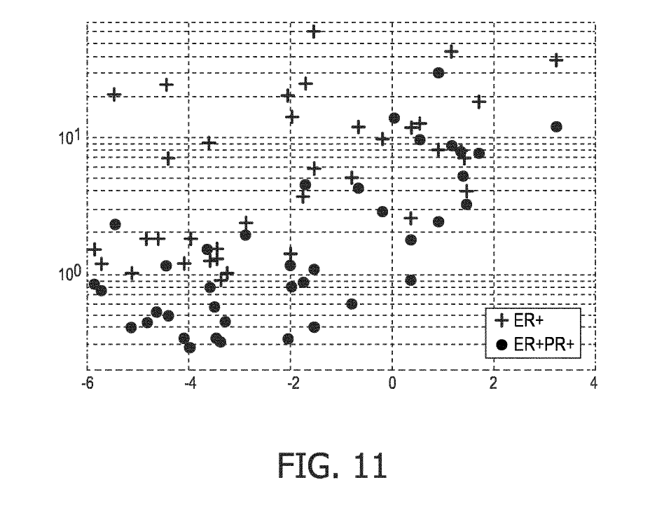

FIG. 11 shows results of experiments performed with 39 breast cancer samples, wherein an ER-only scoring (prior art) based on a single ER staining (abbreviated here as “ER+”) is compared to a scoring that only considers cells of the sample that show both a nuclear presence of the transcription factor, here, ER-α, and a presence of the target gene-encoded protein, here, PR, based on a first staining and a second staining (abbreviated here as “ER+PR+”), as described above. In the graph, the log 2 of the probability—inferred by means of the method described in Verhaegh W. et al. (ibid)—that the signal transduction pathway, here, the ER-α signal transduction pathway, is active in the respective sample, is indicated on the x-axis. More positive values of the log 2 of the probability (right side of the graph) thereby indicate a higher likelihood that the ER-α signal transduction pathway is active in the sample whereas more negative values (left side of the graph) indicate that the ER-α signal transduction pathway is more likely to be inactive in the sample. The y-axis indicates the percentage of cells found according to the prior art for “ER+” and by means of the methods according to the invention for “ER+PR+”, respectively.

The results show that with the conventional ER staining and hospital protocols, as they are used in the prior art, a number of the samples are incorrectly found to indicate an activity of the ER-α signal transduction pathway (top left quadrant of the graph). These “false positives” show a high percentage of ER positive cells; however, the log 2 of the probability indicates that the ER-α signal transduction pathway is actually more likely to be inactive in the respective sample. If a patient with such an ER positive result was treated with hormonal therapy, for instance, neoadjuvant, adjuvant and/or metastatic therapy, it is quite likely that he/she may not show the desired response to the therapy. In contrast, for “ER+PR+”, we find that the determined percentages of cells that show both a nuclear presence of ER-α (i.e., the transcription factor) and a presence of PR (i.e., the target-gene encoded protein) qualitatively increase with an increasing log 2 of the probability that the transcription factor is active. This may allow reducing the number of “false positives” compared to the prior art and providing a higher specificity in assessing activity of the ER-α signal transduction pathway. The threshold for positivity was determined in two different ways, which result in very similar outcomes. In the first variant, negative controls, i.e., stainings where the primary antibodies were left out of the assay and only the secondary antibodies were added to the tissue slice, were stained. Of these negative controls, the mean nuclear intensity was determined and the intensity where 99% of the cells are below was chosen as the cut-off point for calling a cell in the normally stained samples positive. In the second, alternative variant, the cytoplasmic staining was used to determine the background locally. This was done to compensate for the highly variable background staining across individual samples. To label a cell as positive, it should have significantly higher nuclear total cytoplasmic staining ratio than the negative controls.

In the first to third embodiment of a method for inferring activity of a transcription factor or a signal transduction pathway, here, the ER-α signal transduction pathway, in a subject described with reference to FIGS. 1 to 3, a counterstain with a wavelength that suitably contrasts the wavelengths of the stains from the first staining and the second staining can additionally be performed in order to make the stained structures of interest, here, cells and their nuclei, more easily detectable. In this case, it can also be possible that the outlines of cells and their nuclei are detected based on the color information from the counterstain. One particularly suitable substance for performing such a counterstain could be, for instance, Hematoxylin and/or Eosin (in the case of a conventional brightfield staining). In the case of a fluorescent staining, DAPI or Hoechst are suitable options.

While the present invention has been described above for a case where the signal transduction pathway is the ER-α signal transduction pathway, the transcription factor is ER-α, the target gene is PgR and the target-gene encoded protein is PR as well as where the subject is a medical subject, in particular, a cancer patient, more particularly, a breast cancer patient, the present invention is also highly relevant for other cases. For instance, in addition to the estrogen receptor, the androgen receptor, the progesterone receptor, the glucocorticoid receptor, the retinoic acid receptor (RAR), the peroxisome proliferator-activated receptor (PPAR), the vitamin D receptor (VDR), and the orphan nuclear receptor, the present invention can be used with the Wnt transcription factor, the Hedgehog (HH) transcription factor, the transforming growth factor beta (TGF-β) transcription factor, the Notch transcription factor, the NF-κB transcription factor, the phospoinositide 3-kinease (PI3K) transcription factor, the activating protein-1 (AP1) transcription factor, the janus kinase/signal transducers and activators of transcription (Jak-STAT) transcription factor and all transcription factors that can switch from an inactive to an active gene transcribing mode, in combination with at least one transcription-specific target-gene encoded protein. Since these pathways play a role in all types of cancers, the present invention can be applied to all cancer types in all organs, as well as all non-malignant tumors and other diseases with abnomal tissue pathology when biopsied, such as, liver (e.g., primary liver cancer, metastatic liver cancer, cirrhosis), lung (e.g., primary and metastatic cancer, lung fibrosis), kidney (e.g., Wilms tumor, acute glomerulonephritis), bladder (e.g., bladder carcinoma, epithelial hyperplasia), gastro-intestinal (e.g., gastric en esopageal cancer, colon adenoma, inflammatory bowel disease), heart (e.g., cardiomyopathy, cardiac tumors), uterus (e.g., endometrial cancer, endometriosis, fibromas), ovariae (e.g., ovarian cancer, hyperplastic ovarial syndrome), prostate (e.g., prostate hyperplasia, prostate carcinoma, prostatitis), testes (e.g., testis carcinoma), muscle (e.g., sarcomas), skin (e.g., melanoma, squamous carcinoma, eczema), bone (e.g., metastatic tumor, leukemia, osteosarcoma), oral (e.g., mucositis, head and neck cancer), brain (e.g., glioma, Creutsfeld-Jacob), blood vessels (e.g., atherosclerosisr), and fibrous tissue (e.g., fibromas and fibrosarcomas, lung and bladder fibrosis, scar tissue) diseases.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. For instance, while in the embodiment of the system for use in inferring activity of a transcription factor of a signal transduction pathway in a subject described with reference to FIG. 6, the quantifying unit 33 and the (optional) inferring unit 34 are described/shown as two separate units, they may also be realized as a single unit.

A computer program may be stored/distributed on a suitable medium, such as an optical storage medium or a solid-state medium, supplied together with or as part of other hardware, but may also be distributed in other forms, such as via the Internet or other wired or wireless telecommunication systems.

Any reference signs in the claims should not be construed as limiting the scope.

The present invention relates to a method for inferring activity of a transcription factor of a signal transduction pathway in a subject. The method comprises performing a first staining for detecting the transcription factor in cells of a sample of the subject and performing a second staining for detecting a protein encoded by a target gene of the transcription factor in cells of the sample. The method further comprises quantifying cells of the sample that show both a nuclear presence of the transcription factor and a presence of the target gene-encoded protein based on the first staining and the second staining, and inferring the activity of the transcription factor in the subject based on the quantifying. This allows the presented method to more reliably infer the activity of the transcription factor in the subject.

1. A method for inferring activity of a transcription factor of a signal transduction pathway in a subject, comprising:
performing a first staining for detecting the transcription factor in cells of a sample of the subject,
performing a second staining for detecting a protein encoded by a target gene of the transcription factor in cells of the sample,
quantifying cells of the sample that show both a nuclear presence of the transcription factor and a presence of the target gene-encoded protein based on the first staining and the second staining, and
inferring the activity of the transcription factor in the subject based on the quantifying.

2. The method according to claim 1, wherein the first staining and the second staining are performed on a same slide of the sample.

3. The method according to claim 1, wherein the first staining and the second staining are performed on different slides of the sample,
wherein the quantifying comprises spatially registering a digital image of the slide of the first staining and a digital image of the slide of the second staining, such that on both slides corresponding cells can be detected.

4. The method according to claim 3, wherein the different slides are obtained from adjacent cross-sections of the sample or from cross-sections of the sample that are located in close proximity to each other in the sample.

5. The method according to claim 1, wherein the first staining and/or the second staining is/are performed in the form of an assay selected from the group consisting of (i) an immunohistochemistry (IHC) assay, (ii) an immunofluorescent assay, and (iii) another staining assay based on a high affinity binding to the transcription factor or the target gene-encoded protein.

6. The method according to claim 5, wherein the quantifying comprises:
determining, from a selected population of the cells of the sample, a percentage of cells that show both the nuclear presence of the transcription factor and the presence of the target gene-encoded protein, and/or
determining, from a selected population of the cells of the sample, cells that show the nuclear presence of the transcription factor and determining, from the determined cells, a percentage of cells that also show the presence of the target gene-encoded protein.

7. The method according to claim 6, wherein the population is a population of cancer cells, in particular, breast cancer cells.

8. The method according to claim 2, wherein the quantifying comprises analysing at least one digital image of the same slide of the first and the second staining or the digital images of the slide of the first staining and the slide of the second staining using computer-implemented image analysis techniques.

9. The method according to claim 1, wherein the subject is a medical subject, in particular, a cancer patient, more particularly, a breast cancer patient.

10. The method according to claim 1, wherein the signal transduction pathway is the ER-α signal transduction pathway, the transcription factor is ER-α, the target gene is PgR, and the target gene-encoded protein is PR.

11. A method for assessing the suitability of a therapy for a subject, the therapy being directed towards a signal transduction pathway, comprising:
performing the method as defined in claim 10 for inferring activity of a transcription factor of the signal transduction pathway in the subject, and
assessing the suitability of the therapy based on the inferred activity,
or
a method for stratifying a subject, comprising:
performing the method as defined in claim 10 for inferring activity of a transcription factor of a signal transduction pathway in the subject, and
stratifying the subject based on the inferred activity.

12. A system for use in inferring activity of a transcription factor of a signal transduction pathway in a subject, comprising:
a first staining kit for performing a first staining for detecting the transcription factor in cells of a sample of the subject,
a second staining kit for performing a second staining for detecting a protein encoded by a target gene of the transcription factor in cells of the sample,
a quantifying unit for quantifying cells of the sample that show both a nuclear presence of the transcription factor and a presence of the target gene-encoded protein based on the first staining and the second staining, and
optionally, an inferring unit for inferring the activity of the transcription factor in the subject based on the quantifying.

13. A system for use in assessing the suitability of a therapy for a subject, the therapy being directed towards a signal transduction pathway, comprising:
the system for inferring activity of a transcription factor of the signal transduction pathway in the subject as defined in claim 12, and
optionally, an assessing unit for assessing the suitability of the therapy based on the inferred activity,
or
a system for use in stratifying a subject, comprising:
the system for use in inferring activity of a transcription factor of a signal transduction pathway in the subject as defined in claim 12, and
optionally, a stratifying unit for stratifying the subject based on the inferred activity.

14. A computer program for use in inferring activity of a transcription factor of a signal transduction pathway in a subject, the computer program comprising program code means for carrying out the quantifying step and, optionally, the inferring step of the method for use in inferring activity of the transcription factor in the subject as defined in claim 1, when the computer program is run on a computer,
or
a computer program for use in assessing the suitability of a therapy for a subject, the therapy being directed towards a signal transduction pathway, the computer program comprising program code means for carrying out the quantifying step and, optionally, the inferring step and/or the assessing step of the method for use in assessing the suitability of the therapy for the subject when the computer program is run on a computer,
or
a computer program for use in stratifying a subject, the computer program comprising program code means for carrying out the quantifying step and, optionally, the inferring step and/or the stratifying step of the method for use in stratifying the subject, when the computer program is run on a computer.
